(19) United States
(12) Patent Application Publication
(10) Pub. No.: US 2026/0053946 A1
(43) Pub. Date: Feb. 26, 2026

(54) OXIDIZED CARBON PEG-OAC NANOZYMES FOR MITOCHONDRIAL DISORDERS

(71) Applicant: The Texas A&M University System, College Station, TX (US)

(72) Inventors: Thomas A. KENT, Houston, TX (US); Paul J. DERRY, Houston, TX (US)

(73) Assignee: The Texas A&M University System, College Station, TX (US)

(21) Appl. No.: 19/307,712

(22) Filed: Aug. 22, 2025

Related U.S. Application Data
(60) Provisional application No. 63/686,547, filed on Aug. 23, 2024.

(51) Int. Cl. A61K 47/69; A61K 9/00; A61K 47/02; A61K 47/54; A61P 25/28

(52) U.S. Cl. CPC A61K 47/6935; A61K 9/0019; A61K 47/02; A61K 47/547; A61P 25/28

(57) ABSTRACT

The present invention is directed to a composition and a method for treating a mammal exhibiting an inherited mitochondrial disease. That method comprises administering a pharmaceutical composition containing a mitochondria-treating effective amount of PEG-OAC nanozymes, DEF-OAC-PEG nanozymes or both nanozymes dissolved or dispersed in a physiologically tolerable diluent. In that composition, PEG is an acronym for a reacted alpha-amino-omega-methoxy-poly(ethylene glycol) substituent, OAC is an acronym for oxidized activated charcoal particle, and DEF is an acronym for a reacted deferoxamine substituent. Both of the PEG and the DEF substituents are each covalently bonded to the OAC particle by the primary amino group on each.

### CROSS-REFERENCE TO RELATED APPLICATIONS

This application claims the benefit of and priority to U.S. Provisional Application No. 63/686,547 filed on Aug. 23, 2024, the content of which is incorporated by reference in its entirety.

### STATEMENT OF GOVERNMENT LICENSE RIGHTS

This invention was made with government support under grant numbers R01NS094535 and N21NS084290, awarded by the National Institutes of Health. The government has certain rights in the invention. This invention was also made with the support of the Welch Foundation under grant number BE-0048.

### BACKGROUND

Mitochondrial diseases such as Friedreich's ataxia (FRDA) are rare but have life-long consequences on day-to-day function with a significant loss of quality of life and longevity. Several mitochondrial diseases are directly or indirectly caused by genetic mutations as is FRDA, some of which are in the cellular nucleus and others are in the mitochondrial genes (mtDNA) themselves. Such disorders are referred to herein as inherited or genetic mitochondrial diseases.

Inherited or genetic mitochondrial diseases include but are not limited to Mitochondrial Encephalopathy with Lactic Acidosis and Stroke-Like Episodes (MELAS), Leigh's syndrome, Myoclonic epilepsy with ragged red fibers (MERRF), and Leber hereditary optic neuropathy (LHON). These diseases have intrinsic genetic mutations in mtDNA-located genes that prevent the synthesis of important mitochondrial proteins resulting in a reduction of oxidative phosphorylation. 80% of all MELAS cases are due to a mutation of the mtDNA gene m.3243 that results in the loss of translation of tRNA-Leucine and interrupts protein synthesis.

FRDA is a mitochondrial disease associated with the loss of an essential mitochondrial protein. FRDA is an autosomal recessive disorder characterized by trinucleotide repeats in the autosomal DNA gene FXN. It affects approximately 1 in 50,000 live births in the USA, with almost all patients developing cardiomyopathy in addition to ataxia. FRDA arises from the pathogenic expansion of GAA repeats in the FXN gene, leading to a decrease in frataxin protein levels. Individuals with FRDA exhibit over 200 GAA repeats, in contrast to healthy individuals who have fewer than 40 GAA repeats.

Symptoms of the disease typically emerge in early childhood and include visual impairments, pronounced gait and limb ataxia, muscle weakness, diabetes mellitus, and a heightened risk of cardiovascular conditions like hypertrophic cardiomyopathy. In later stages, heart disease becomes the primary cause of mortality, typically occurring in the fifth or sixth decade of life.

FRDA is an autosomal recessive trinucleotide repeat disorder characterized by visual impairments, pronounced gait and limb ataxia, muscle weakness, diabetes and heart failure. FRDA arises from the pathogenic expansion of GAA repeats in the FXN gene, leading to a decrease in frataxin protein levels.

FRDA predisposes to iron mediated cell death pathway, ferroptosis. Propensity to ferroptosis can be influenced by another cellular phenotype, senescence. Although senescence is typically associated with aging, numerous cellular stressors including dysfunctional mitochondria as in FRDA, can initiate halting of the cell cycle and expression of senescence phenotype. A prominent feature is lysosomal enlargement and inability to phagocytize iron so that intracellular iron concentrations reach levels high enough to initiate ferroptosis, yet cells become ferroptosis resistant.

Early or incomplete senescence may predispose to ferroptosis. Persistent senescence disrupts cell function and can transition to an inflammatory phenotype, that can recruit and detrimentally affect neighboring cells. FRDA literature with respect to excess iron and clinical outcomes reports for iron chelation therapy appears inconsistent, which could be explained by different stages of ferroptosis and senescence.

There is a need for treating inherited or genetic mitochondrial diseases beyond the current treatments. Disclosed herein are methods for treatment of inherited or genetic mitochondrial diseases comprising PEG-OAC nanozymes, DEF-OAC-PEG nanozymes or combinations thereof.

### BRIEF SUMMARY OF THE INVENTION

The present disclosure teaches a composition and method for treating inherited or genetic mitochondrial diseases. The present disclosure teaches treatments that prolong and/or enhance the quality of life of those mammals having such a disease by assisting their mitochondria in producing alternative energy sources that can assist their life processes. In one embodiment, the mammal is a human. In one embodiment, the human is a patient. In one embodiment the term nanozyme means pleozymes

In one embodiment, a method for treating a mammal presenting with an inherited mitochondrial disease is taught. In one embodiment a method comprising administering a pharmaceutical composition comprising a mitochondria-treating effective amount of composition from the group consisting of PEG-OAC nanozymes, DEF-OAC-PEG nanozymes or combinations thereof. In one embodiment, both nanozymes are dissolved or dispersed in a physiologically tolerable diluent. The composition PEG is an acronym for a reacted alpha-amino-omega-methoxy-poly(ethylene glycol) substituent, OAC is an acronym for oxidized activated charcoal particle, and DEF is an acronym for a reacted deferoxamine substituent. In one embodiment, the PEG and DEF substituents are each covalently bonded to the OAC particle by the primary amino group on each of the PEG and the DEF substituents.

In one embodiment, the PEG substituents have an average molecular weight of about 2000 to about 10,000 Da. In one embodiment, the average molecular weight of the PEG substituents is about 3000 to about 7000 Da. In one embodiment, the average molecular weight of the PEG substituents is about 4000 to 6000 Da. In one embodiment, the average molecular weight of the PEG substituents is about 5000 Da.

In one embodiment, the PEG-OAC nanozymes, DEF-OAC-PEG nanozymes can contain about the same number of PEG substituents or very different numbers, depending on the synthesis used. Following the synthetic route of FIG. 1, when equal molar amounts of DEF and PEG reactants are used, the resulting DEF-OAC-PEG nanozymes generally tend to have about equal numbers of PEG substituents. A skilled worker will understand the appropriate synthetic procedures and reactant ratios for obtaining a desired product with little difficulty. In some embodiments, only one of the nanozymes is present.

In one embodiment, the PEG-OAC nanozymes contain an average of about 2 to about 5 of said reacted PEG substituents per particle. In one embodiment, an average of about 2 to about 5 PEG groups are chemically bonded to the particle. In one embodiment, about 2 to about 5 PEGs in PEG-OAC (without DEF). In one embodiment, an average of about 2 to about 5 PEG groups are chemically bonded to the particle and about 10 to about 20 DEF substituents are chemically bonded to the particle. In one embodiment, about 2 to about 5 PEGs in PEG-OAC-DEF. In one embodiment, an average of about 2 to about 5 PEG substituents per particle.

In one embodiment, the physiologically tolerable diluent is an aqueous liquid adapted for parenteral administration. Illustrative aqueous liquid diluents include Ringer's solution, isotonic sodium chloride solution or phosphate-buffered saline. In another embodiment, the physiologically acceptable diluent is adapted for oral administration. As such, the diluent can be an aqueous liquid or a solid. In other embodiments, the pharmaceutical composition is formulated as a solid for oral administration.

In one embodiment, the administration of the composition is repeated, and those repeats can be in a single day, over several days, months or years. In one embodiment, the dosage is an amount of about 0.1 to about 2 mg/kg. In one embodiment, the dosage is an amount of about 0.05 to about 4 mg/kg. In one embodiment, the dosage is an amount of about 0.5 to about 1.5 mg/kg. In one embodiment, the dosage is an amount of about 0.1 to about 1 mg/kg.

In one embodiment, the mitochondrial disease or disorder is selected from the group consisting of FDRA, Mitochondrial Encephalopathy with Lactic Acidosis and Stroke-Like Episodes (MELAS), Leigh's syndrome, Myoclonic epilepsy with ragged red fibers (MERRF), and Leber hereditary optic neuropathy (LHON).

### BRIEF DESCRIPTION OF THE DRAWINGS

Non-limiting embodiments will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the drawings forming a portion of this disclosure.

FIGS. 1A-H, illustrate several facets of the synthesis and characterization of DEF-OAC-PEG nanozymes used in this invention; FIG. 1A is a reaction scheme for the synthesis of DEF-OAC-PEG nanozymes from OACs derived from fuming nitric acid oxidation of sieved cocoanut shell-derived activated charcoal; FIG. 1B is a representative high-resolution transmission electron microscopy of OACs (scale bar 20 nm); FIG. 1C is a graph illustrating the size distribution of cOAC particles (mean: 3-3.5 nm; maximum: 7 nm; n=360 particles); FIG. 1D illustrates a thermogravimetric analysis of OACs showing a mass loss of 56% (dashed line), PEG-OACs nanozymes (midtone line; 91%), and DEF-OAC-PEG nanozymes showing a mass loss of 78% (black line); FIG. 1E is a graph showing nanoparticle tracking analysis of PEG-OACs (midtone line; mean: 89 nm; mode: 78 nm) and DEF-OAC-PEG (black line; mean: 94 nm; mode: 89 nm); and FIGS. 1F-1H show high resolution X-ray photoelectron spectroscopic C 1s spectra of OAC, PEG-OAC, and DEF-OAC-PEG nanozymes, respectively.

FIGS. 2A-B, illustrate the effects of PEG-OAC and DEF-OAC-PEG nanozymes on mitochondrial respiration in which FIG. 2A illustrates the oxygen consumption rate (OCR) plot of bEnd.3 cells treated with untreated cells as a control (CTL), and cells treated with PEG-OAC or DEF-OAC-PEG nanozymes (4 mg/mL) showing enhanced OCR for both particle types; FIG. 2B shows that following the injection of oligomycin, an ATP synthase inhibitor for Complex V, the OCR was higher with particle treatment than without.

FIG. 3 is a schematic depiction of a contemplated nanozyme and the known enzyme-like actions nanozyme materials carry out. Including multiple reaction pathways facilitated by PEG-OAC or DEF-OAC-PEG nanozymes including NADH oxidation to NAD+, H2S oxidation to HSSH and S2O32-, and superoxide dismutation.

FIGS. 4A-B, are graphs that illustrate the differences in maximal oxygen consumption rate in healthy (isogenic) and Friedreich's ataxia (FRDA) cardiomyocytes that have a lower overall maximal oxygen consumption rate than the healthy cells by approximately 60% (FIG. 4A), and wherein FIG. 4B illustrates the change in maximal respiration of isogenic and FRDA cardiomyocytes is shown in FIG. 4B. Here, DEF-OAC-PEG nanozyme treatment increases maximal respiration in FRDA cardiomyocytes by approximately 15-20%.

FIG. 5 is a graph showing the effects of DEF-OAC-PEG nanozyme particles on intracellular iron in bEnd.3 cells and illustrates that when treated with Fe3+, the fluorescent signal from FerroOrange-labeled bEnd.3 cells is lower than without the DEF-OAC-PEG nanozymes, indicating that DEF-OAC-PEG nanozyme treatment reduces intracellular iron levels.

FIG. 6 is a graph that shows the ratios of lipid peroxidation in FRDA fibroblasts using the ratio of the oxidized:reduced C11-BODIPY™ signal (581/591 nm) measured over two image fields.

FIG. 7 shows photomicrographs of FRDAfibroblasts in the assay of FIG. 6 to show the illustrate the effect of no treatment and of two nanozyme types on the oxidized and reduced in which the top row are the reduced C11-BODIPY™ signal and the bottom row are the oxidized C11-BODIPY™ signal in which the brighter top row relative to the bottom row indicates less lipid peroxidation.

FIG. 8 is a graph of the signal intensities in arbitrary units (AU) read from the micrographs of FIG. 9 and other studies.

FIG. 9 displays micrographs that illustrate the effects of omaveloxone (Omav) and the two different nanozymes on the expression of the Nrf2 protein and the translocation of that protein into the cell nucleus compared to an untreated control, the upper row utilizes an fluorescently labeled anti-Nrf2 antibody whereas the lower row is stained with the dye DAPI (4′,6-diamidino-2-phenylindole) a fluorescent dye that binds to DNA and emits blue light when exposed to ultraviolet light.

FIG. 10 is a graph comparing contractility of isogenic (wild type) inducible-pluripotent stem cell-derived cardiomyocytes with those from an FRDA donor.

FIG. 11 is a comparison of cellular senescence indicated by the enzymatic action of b-galactosidase on the molecule X-Gal that produces a dark blue stain in which two micrographs collected from FRDA fibroblasts treated with DEF-OAC-PEG nanozymes for 72 hours or left as a control are shown, and wherein a clear visual difference in the degree of dark staining is shown in the control as opposed to the DEF-OAC-PEG nanozyme-treated cells indicating a reduction in cellular senescence.

FIGS. 12A-D, show cellular senescence plots comparing a FRDA line to two apparently healthy (AHI) fibroblast cell lines over a period of 4 weeks shown in four graphs as FIGS. 12A (one week), 12B (two weeks), 12C (three weeks) and 12D (four weeks), in which cells were treated with PEG-OAC or DEF-OAC-PEG nanozymes twice a week (2 μg/mL for the first two weeks, 4 μg/mL for the last two weeks), and wherein significance between cell types within the week the senescence labeling by X-Gal was measured is shown as asterisks.

FIGS. 13A-C, show Nanozymes (pleozymes) and a positive control that stimulates NAD+. Both increase intracellular NADH in cultured b.End3 cells after 24 hours. FIG. 13A reveals increased NAD+ by both nanozymes (pleozymes) and the positive control that stimulates NAD+, nicotinamide riboside (NIR, suggesting that increased NAD+ is probably consumed after 24 hours in these metabolically active cells that comprise the first line of defense in the blood:brain barrier. FIG. 13B shows that both the nanoparticles and NIR increase NADH, and FIG. 13C shows decreased NAD+/NADH ratio. Mean+standard deviation (both directions). N=5 independent assays. Friedman test. * represents p<0.05.

FIG. 14 shows Nanozymes (pleozymes) increase intracellular ATP levels after 2 and 24 hours in cultured b.End cells in 2 independent assays.

FIG. 15 shows Metabolomics data indicating that nanozymes (pleozymes) enhance intracellular and mitochondrial energetic pathways and catalytically supply metabolic reactions with NAD+ regeneration activities. Localized in the cytoplasm and mitochondria, nanozymes (pleozymes) catalytically accelerate NAD+ regeneration from NADH and subsequent ATP production, leading to an increase in the NAD±-dependent metabolic rates. Nanozymes (pleozymes) also accelerate intracellular energy metabolisms through glycolysis and lactate generation. Nanozyme (pleozyme) treatment also increases fatty acid β-oxidation and supplies acetyl- and succinyl-CoA to the TCA cycle, driving the cycling reactions and replenishing the lactate precursor, pyruvate, from malate.

FIG. 16 shows studies support this mechanism. The energetic model of nanoparticles suggest that Malic Enzyme is mediating at least part of the effect. Here, treatment with the Malic Enzyme 1 inhibitor (ME1 i) 3-(4-(4-hydroxyphenyl)piperazin-1-yl)-1-phenylpyrrolidine-2,5-dione significantly lowered mitochondrial maximal oxygen consumption rate (OCR, y axis), demonstrating that nanoparticles engage enzymes consistent with the above model. While ME1 is primarily cytoplasmic (with limited specificity to ME2 inhibitors other than siRNA), supporting the above model that nanoparticles engage NAD+ dependent pathways that benefit those with mitochondrial disorders.

FIG. 17 shows Increased mitochondrial mass in Friedreich's Ataxia skin fibroblasts due to impaired mitochondrial function which necessitated higher number of mitochondria. Here, mitochondrial mass was quantified by the intensity of Mitotracker® fluorescence mitochondrial probe after 24 hour treatment with PEG-OAC or vehicle in cells from Apparently Healthy individuals (AHI) and Friedreich's Ataxia (FRDA). The basal (CTL) level of mitochondrial signal intensity is higher in FRRDA compared to AHI. The amount of signal reflecting mitochondrial mass is normalized by PEG-OAC treatment in FRDA with no significant difference in AHI.

### DETAILED DESCRIPTION OF THE EMBODIMENTS

The present disclosure teaches a composition and method for treating an inherited or genetic mitochondrial disease.

The FXN gene is located at chromosome locus 9q131, and the pathogenic expansion of GAA leads to a decrease in gene expression and frataxin protein levels. Frataxin, a protein crucial for mitochondrial function, is primarily located in the mitochondrial matrix, where it plays a vital role in regulating energy production, iron metabolism, iron regulation, and the assembly and repair of iron sulfur clusters within the mitochondria. It also regulates the excess of free iron and critical antioxidant mechanisms for cellular protection. There is evidence for ferroptosis, an iron-mediated form of cell death induced by the buildup of lipid peroxides.

Frataxin deficiency interferes with the assembly of iron-sulfur clusters, vital components for the mitochondrial electron transport chain (ETC), a main source for cellular energy through oxidative phosphorylation. ETC disruption causes electron leakage and production of superoxide radical causing oxidative stress and reduced ATP. A misallocation of iron may also promote oxidative stress and ferroptosis. Frataxin mRNA is expressed in high metabolic rate tissues such as liver, neurons and heart, and its deficiency renders these organs exceptionally susceptible to injury.

Cellular senescence contributes to the development and progression of FRDA. The loss of frataxin protein in FRDA leads to increased cellular stress and DNA damage, which are triggers for cellular senescence. Cellular senescence, mitochondrial dysfunction, low energy production, disrupted Ca2+ balance contribute to neurodegeneration and abnormal development in FRDA pathogenesis.

The accumulation of senescent cells can contribute to disease progression by promoting inflammation and impairing tissue function called the senescence-associated secretory phenotype (SASP), which can have detrimental effects on neighboring cells and tissues, including neurons. The relationship between oxidative stress, cellular senescence, and FRDA is targeted for therapeutic approaches to treat the disease.

The SASP phenotype has also been linked to other diseases such as but not exclusive to tumorigenesis in breast cancer, prostate cancer, other carcinomas, melanoma, and cancer-associated effects such as angiogenesis, and affects individual cell types differently. For example, senescent endothelial cells are induced to enhanced motility, higher likelihood of metastasis, and more successful at invasion.

Senescence influences cell death by ferroptosis, but if in a condition in which signals for both exist, inhibiting one can accentuate cell death from the other pathway. Optimum therapy, short of replacing the frataxin protein systemically and in the central nervous system, requires addressing multiple pathological mechanisms simultaneously.

Disclosed herein is a therapeutic strategy to treat mitochondrial diseases, such as Friedreich's ataxia (FRDA), and other mitochondria-related diseases utilizing a 4th generation oxidized carbon-rich molecule with pleiotropic enzymatic actions termed a nanozyme.

To address mechanisms responsible for the interplay between senescence and ferroptosis, taught herein are nanozymes. PEG-OAC nanozymes are derived from harshly oxidized medicinal-grade activated charcoal (PEG-OAC), are unique materials with multiple (pleiotropic) actions that can reduce senescence in hemorrhage models. Carboxylates permit covalently bonding of PEG and an iron chelator, e.g., deferoxamine (DEF), whose entry into cells is greatly facilitated by the nanozyme and in our other works reduces ferroptosis.

PEG-OAC nanozymes catalytically dismutate superoxide, facilitate electron transfer between mitochondrial components and oxidize hydrogen sulfide to antioxidant polysulfides. They localize to the membrane and interior of the cell including co-localization localizing with mitochondria, and exhibit protective effects in acute and chronic disease models including cerebral stroke, traumatic brain injury and metabolic syndrome, without apparent toxicity. The ability of PEG-OAC nanozymes to generate polysulfides elucidates a new mechanism to increase Nrf2 levels: oxidation of Keap1 disulfide bonds, which releases Nrf2 to the nucleus. They improve both mitochondrial and non-mitochondrial energetics.

PEG-OAC nanozymes increase FRDA fibroblast nuclear Nrf2 and enhance proliferation of FRDA-patient derived induced pluripotent stem cells differentiated cardiomyocytes. Proliferation of cardiomyocytes is crucial through young adulthood for normal cardiac function and repair and for neuronal precursors (IPSC-N).

Among FRDA-derived cells from different individuals, there is an inverse relationship between extent of senescence and sensitivity to ferroptosis-mediated cell death. Functional improvement in FRDA-derived cells by PEG-OAC nanozymes vs. deferoxamine-PEG-OAC nanozymes are compared hereinafter. Deficits in proliferation and mitochondrial function of FRDA human induced pluripotent stem cell-(IPSC)-derived neuronal precursors and cardiomyocyte function (e.g., contractility) are improved with treatment with PEG-OAC nanozymes.

The synthesis of a class of oxidized carbon nanoparticles (OCNs) were functionalized with poly(ethylene glycol). This class of materials is synthesized using different starting materials including single-walled carbon nanotubes, anthracitic and bituminous coal, and good manufacturing practice-synthesized coconut-derived activated charcoal. Poly(ethylene glycol)-functionalized hydrophilic carbon clusters (PEG-HCCs), derived from single-walled nanotubes are potent neuroprotectants in traumatic brain injury and ischemic stroke models. The facile synthetic chemistry for functionalizing PEG-HCCs uses conventional amide coupling with DCC and DMAP and is extendable to other amine-containing groups such as deferoxamine which protects against hemorrhagic stroke, and reduce DNA damage through its intrinsic redox mediating actions and/or an additional iron chelation mechanism.

The OCNs are characterized by broad reduction potentials and are capable of rapid catalyzed superoxide dismutation much like superoxide dismutase and acting as an oxidoreductase for NADH, the electron donor for the mitochondrial electron transport chain. In addition to PEG-HCCs, poly(ethylene glycol)-functionalized graphene quantum dots (PEG-GQDs) from bituminous and anthracitic coal were produced and acted as superoxide dismutase mimetics. One oxidized carbon nanoparticle includes the poly(ethylene glycol)-functionalized oxidized activated charcoal (PEG-OAC).

The hydrogen sulfide oxidation mechanism of PEG-HCCs and PEG-OAC nanozymes are disclosed. PEG-OAC nanozymes have differing syntheses, chemistries and physical properties. These nanoparticles have the quality of being pleiotropic because of the multitude of chemical mechanisms facilitated by the nanoparticles and are common throughout. Because of this, taught herein are potential disease targets based on these chemical mechanisms for diseases including Down Syndrome or a disease of mitochondria such as Friedreich Ataxia.

Multiple therapeutic approaches are needed to maximize benefit given the multiple FRDA pathogenesis mechanisms and injuries prior to diagnosis.

Disclosed herein are new mechanisms to increase Nrf2 levels given the ability of PEG-OAC nanozymes to generate polysulfides: oxidation of Kelch-like ECH-associated protein 1 (Keap1) disulfide bonds, which releases Nrf2 to the nucleus. Targeting a single pathway is not optimally effective in many conditions.

For example, in models of intracerebral hemorrhage, predecessors to PEG-OAC nanozymes called poly(ethylene glycol)-conjugated hydrophilic carbon clusters (PEG-HCC), were able to address both senescence and ferroptosis by covalently bonding the iron chelator deferoxamine to the PEG-HCC (DEF—HCC-OAC) using 100× lower doses of deferoxamine than when deferoxamine is used alone, facilitated by the avid cellular uptake of the parent particle whereas deferoxamine alone has inconsistent uptake requiring high doses that are toxic to some cells. In one embodiment, the disclosure teaches a method for treating a mammal presenting with an inherited or genetic mitochondrial disease A non-limiting list of those diseases include examples such as Friedreich's ataxia (FRDA), Mitochondrial Encephalopathy with Lactic Acidosis and Stroke-Like Episodes (MELAS), Leigh's syndrome, Myoclonic epilepsy with ragged red fibers (MERRF), and Leber hereditary optic neuropathy (LHON). These diseases have intrinsic genetic mutations in mtDNA-located genes that prevent the synthesis of important mitochondrial proteins resulting in a reduction of oxidative phosphorylation. 80% of all MELAS cases are due to a mutation of the mtDNA gene m.3243 that results in the loss of translation of tRNA-Leucine and interrupts protein synthesis.

Another such example is Leigh Syndrome where many of the Complex I subunit genes are encoded into chromosomes. Leigh Syndrome has a similar bioenergetic failure due to an inability to oxidize NADH by Complex I and transport electrons to Complex IV.

FRDA is used herein as an exemplar of the group of inherited or genetic mitochondrial diseases.

In one embodiment, the method comprises administering a pharmaceutical composition containing a mitochondria-treating effective amount of PEG-OAC nanozymes, DEF-OAC-PEG nanozymes or both nanozymes dissolved or dispersed in a physiologically tolerable diluent. In that composition PEG is an acronym for a reacted alpha-amino-omega-methoxy-poly(ethylene glycol) substituent, OAC is an acronym for oxidized activated charcoal particle, and DEF is an acronym for a reacted deferoxamine substituent. The PEG and DEF substituents are each covalently bonded to the OAC particle by the primary amino group on each of the PEG and the DEF substituents.

In one embodiment, the PEG substituents have an average molecular weight of about 2000 to about 10,000 Da. In one embodiment, the average molecular weight of the PEG substituents is about 3000 to about 7000 Da. In one embodiment, the average molecular weight of the PEG substituents is about 4000 to 6000 Da. In one embodiment, the average molecular weight of the PEG substituents is about 5000 Da.

In one embodiment, the PEG substituents are prepared using alpha-amino-omega-methoxy-poly(ethylene glycol) of a desired molecular weight as the reactant. These materials are also known in the art as alpha-methoxy-omega-amino-poly(ethylene glycol). They are available in several molecular weights from several suppliers including Iris Biotech GmbH, Marktredwitz, Germany.

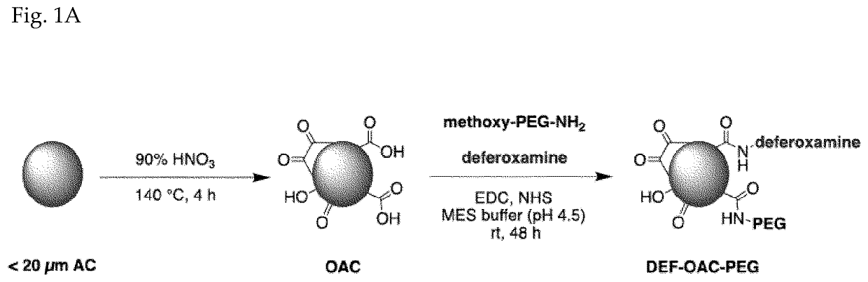

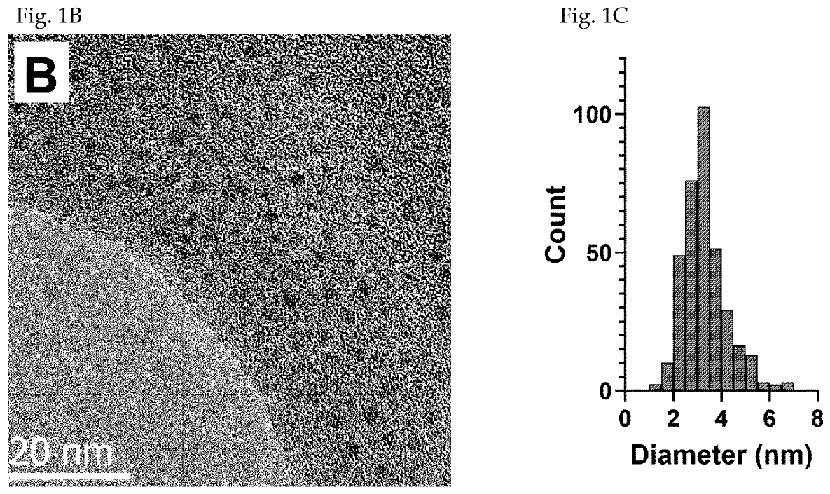

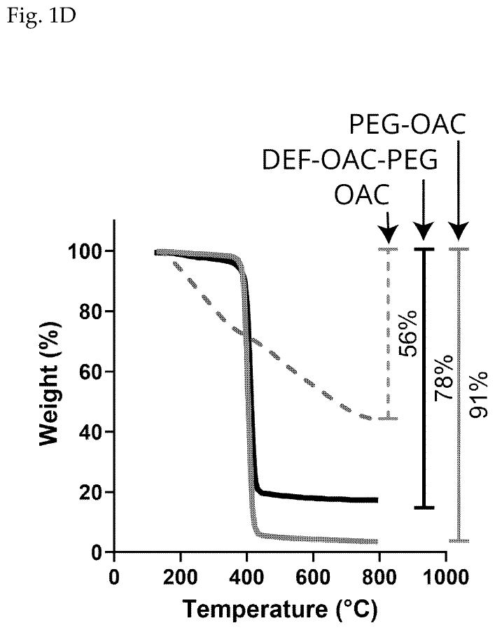

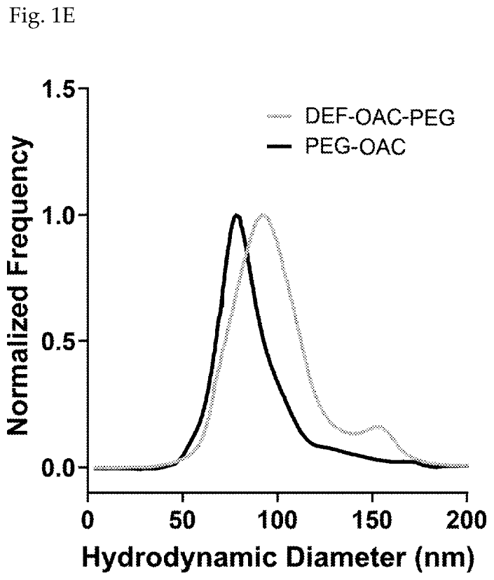

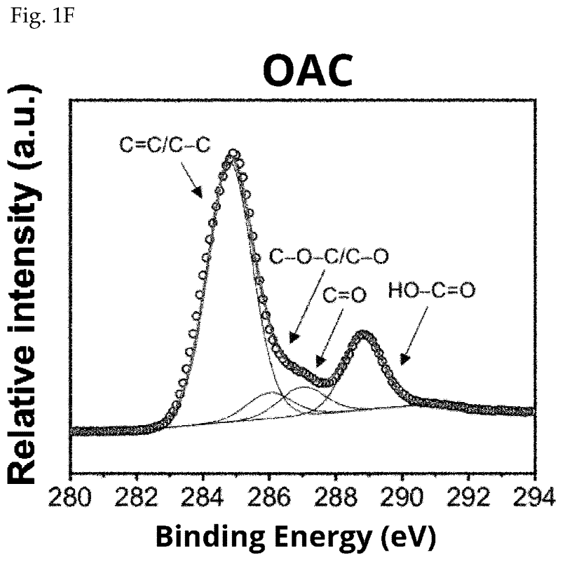

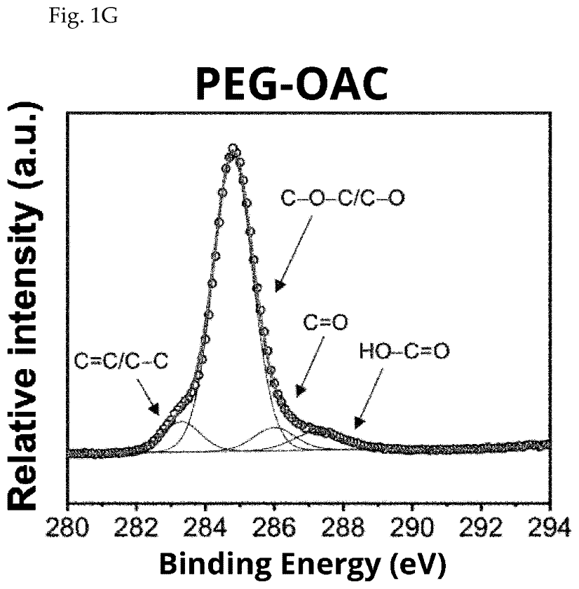

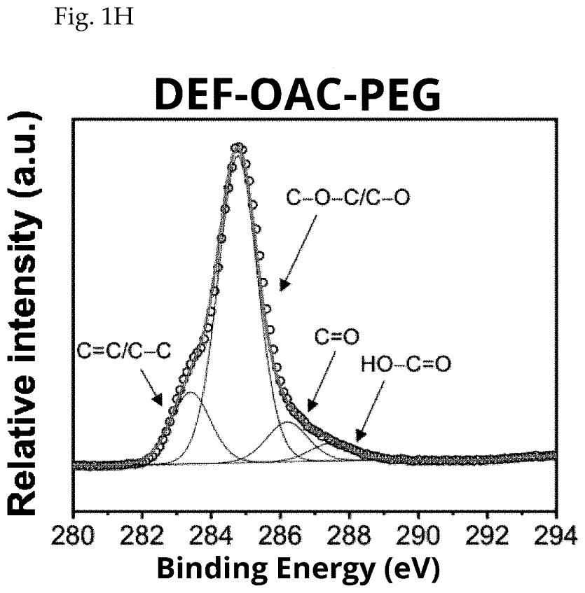

The PEG-OAC nanozymes, DEF-OAC-PEG nanozymes can contain about the same number of PEG substituents or very different numbers, depending on the synthesis used. Following the synthetic route of FIG. 1, when equal molar amounts of DEF and PEG reactants are used, the resulting DEF-OAC-PEG nanozymes have tended to have about similar numbers of PEG and DEF substituents. However, more recent work indicates that the primary amine of the PEG reactant is somewhat less reactive than the primary amine of the DEF reactant, with there being an average of 2 to about 5 PEG groups chemically bonded to the particle and about 10 to about 20 DEF substituents chemically bonded to the particle. The PEG and DEF substituents are typically bonded to the OAC particle using amide-forming chemistry using the particle's carboxyl groups and the substituents' primary amines.

If the PEG-OAC particle is made first and then reacted with the DEF reactant, the PEG-OAC and DEF-OAC-PEG nanozymes have the same average number of PEG substituents per particle. A skilled worker will understand the appropriate synthetic procedures and reactant ratios for obtaining a desired product with little difficulty.

In one embodiment, only one of the nanozymes is present. That is particularly the case when the nanozyme is PEG-OAC. When synthesis is directed to DEF-OAC-PEG, there is a possibility of there being some unreacted PEG-OAC being present along with the DEF-OAC-PEG.

In one embodiment, the physiologically tolerable diluent is an aqueous liquid adapted for parenteral administration. In one embodiment, the physiologically acceptable aqueous liquid diluent is adapted for oral administration. In one embodiment, the pharmaceutical composition is a solid that is adapted for oral administration so that the diluent is typically a solid. Thus, the diluent can be a liquid or a solid.

In one embodiment, the administration is repeated, and those repeats can be in a single day, over several days, months or years. In one embodiment, the dosage is an amount of about 0.1 to about 2 mg/kg.

### EXAMPLES

### I. Effect of the Invention on Friedreich's Ataxia

Friedreich's ataxia (FRDA) is a rare genetic disorder hallmarked by a trinucleotide (GAA) repeat expansion in the frataxin gene (FXN). FRDA effects approximately 1:50,000 live births with symptoms such as cardiomyopathy, visual impairments, difficulty walking, weakness and diabetes. The trinucleotide repeats of mutant FXN lead to reduced transcription efficiency and an overall loss of FXN production.

Although FRDA is a genetic disease, it also shares several generic characteristics of other mitochondrial diseases that permits its use as a model for other mitochondrial diseases.

FXN is an important element of iron metabolism, and a loss of its expression leads to a reduction in mitochondrial iron-sulfur clusters which are essential for oxidative phosphorylation. Disruption of the electron transport chain leads to an accumulation of superoxide and other reactive species in the mitochondria and further reduces their energy conversion efficiency.

FRDA has multiple disease endpoints based on its pathophysiology. Cells that produce substantial amounts of frataxin are most susceptible to damage which mainly consists of neurons, cardiac and skeletal myocytes, and peripheral nerves. The iron accumulation and runaway reactive oxygen species (ROS) cascade in mitochondria is an important element to the pathology of FRDA, but other pathways also exist such as ATP is depletion or senescence in affected tissues.

Therefore, targeting any one pathway may not ultimately be as effective as targeting multiple pathways simultaneously either as a single drug or as an adjuvant to a second drug. Some examples include reducing oxidative stress through an antioxidant mechanism chelation of iron using a specific chelator, upregulating the expression of antioxidant enzymes by activating Nrf2, or increasing mitochondrial biogenesis or increasing both mitochondrial and non-mitochondrial bioenergetics. Disclosed herein are ways that pleiotropic oxidized carbon nanoparticles may facilitate some of these treatment strategies.

### A. Iron Chelation

Without FXN, iron is misallocated and may lead to ferroptosis, particularly in energetically active tissues such as the liver, brain, and muscles. One biochemical hallmark of ferroptosis is the appearance of lipid peroxides.

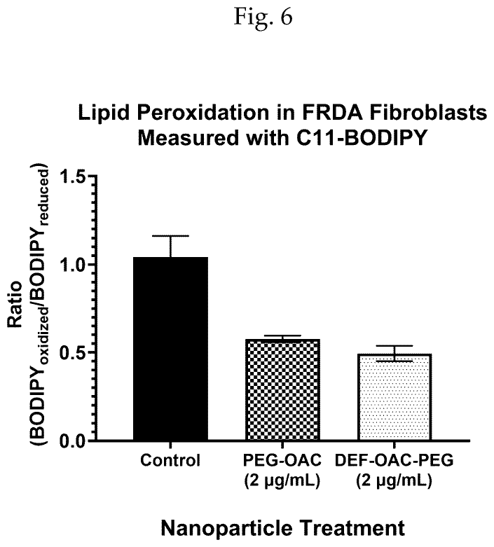

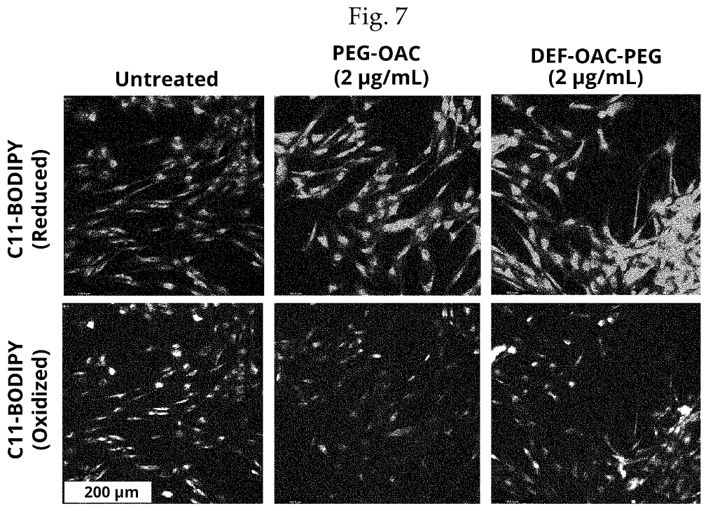

Using lipid peroxidation as a marker in FRDA, we tested the effects of PEG-cOAC and DEF-OAC-PEG nanozymes on FRDA fibroblasts and we found that lipid peroxidation, measured using a peroxidation-sensitive C11-BODIPY ratiometric probe assay, was reduced in the particle treated cells as indicated by a reduction in the oxidized:reduced C11-BODIPY signal (FIG. 6). Example micrographs of the imaged cells are shown in FIG. 7.

FIG. 7 indicates lipid peroxidation (one feature of ferroptosis) by presence of higher green (bottom row of FIG. 7) fluorescence relative to red (top row of FIG. 7), with the opposite for reduced lipid peroxidation. In FRDA-primary human fibroblast cells, DEF-OAC-PEG nanozyme treatment induced a predominance of red fluorescence with trends to higher red with the DEF-OAC-PEG nanozymes as would be predicted if iron were involved in lipid peroxidation.

Nuclear Factor Erythroid 2-related factor 2 (Nrf2) is an antioxidant response transcription factor which under oxidative stress relocates to the nucleus and binds on Antioxidant Response Element promoter regions. Normally, Nrf2 is bound to Keap1, however oxidation of the cysteine residues on Keap1 lead to Nrf2 becoming unbound and active as a transcription factor in the nucleus. The genes associated with Nrf2 activation are highly varied, however many are related to antioxidant enzymes such as: glutamate-cysteine ligase catalytic and modulator subunits (GCLC and GCLM), glutathione synthetase (GSS), thioredoxin, thioredoxin reductase, sulfiredoxin, peroxiredoxin, glutathione peroxidase, superoxide dismutase 1 (SOD1), catalase, and glutathione reductase.

Nrf2 expression in FRDA is downregulated, leading to a reduction in antioxidant response element gene transcription. Increasing the amount of free Nrf2 may have beneficial therapeutic effects in FRDA.

Omaveloxone (Omav) is an FDA-approved treatment for FRDA and is currently available on the open market. Omav is an inhibitor of Nrf2 ubiquitinylation that prolongs its active time in the cell and increases the activation of ARE-promoted genes. The treatment showed a significant reduction in neurological function decline in 40 study participants. An extended duration study of 72 weeks showed continuous reduction in neurological decline in the treatment arm of the study.

DEF-OAC-PEG nanozymes increase levels of Nrf2 in the cell which may have a synergistic effect with existing commercial therapies such as SKYLARYS® (omaveloxolone) that also act on Nrf2 but likely through a different pathway. One way to activate the above pathway for Nrf2 is by reducing the disulfide bonds holding Nrf2 to Keap1, here we used lipoic acid (LA).

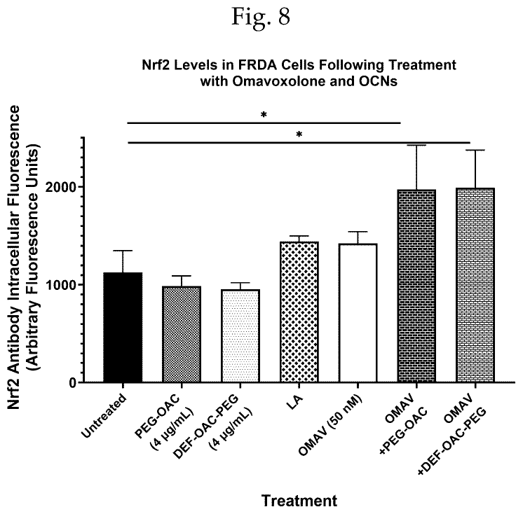

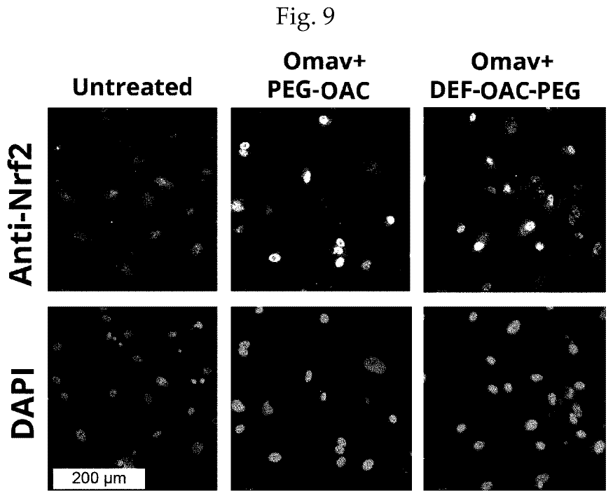

There was no increase in Nrf2 levels in FRDA cardiomyocytes with the particles on their own (FIG. 8) and found that the change in Nrf2 levels was the same between lipoic acid (LA) treatment and treatment with omaveloxolone (FIG. 8). However, when treatment separately included each nanozyme and omaveloxolone, there was an incremental effect in Nrf2 levels that was not seen with the DEF-OAC-PEG nanozymes or PEG-OAC nanozymes alone, suggesting an adjuvant effect (FIG. 8). Fluorescence microscopy of anti-Nrf2 labeled cells showed a higher degree of nuclear translocation of Nrf2 in the Omav+nanozyme treated cells than in the untreated control (FIG. 9)

### B. PEG-OAC Nanozymes Increase Cellular ATP

Cellular ATP production is an important metric of mitochondrial function. The vast majority of ATP produced by a cell comes from oxidative phosphorylation in the mitochondria, however some also comes from glycolysis in the cytosol. Because of prior work that showed that PEG-HCC nanozymes may enhance the activity of the electron transport chain, we tested the hypothesis that ATP production was also enhanced as well.

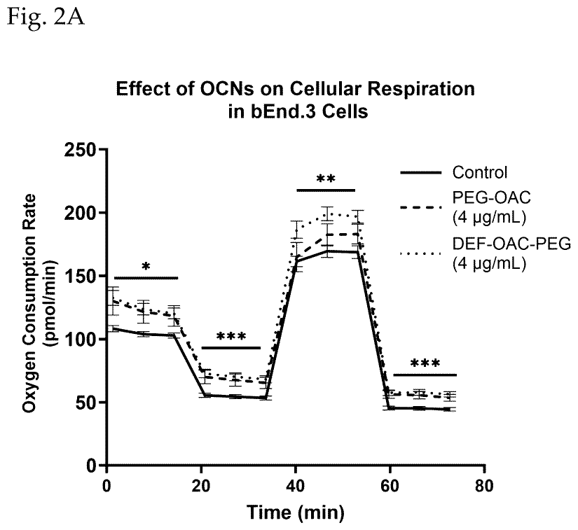

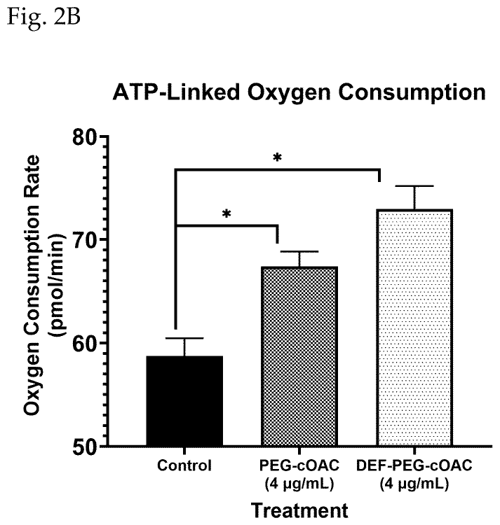

Here, we treated bEnd.3 cells with 4 mg mL-1 PEG-OAC nanozymes for 24 hours and performed a MitoStress Assay to collect changes to the oxygen consumption rate (OCR; FIG. 2A), and ATP production via ATP-linked oxygen consumption (FIG. 2B). We found a significant increase in oxygen consumption in cells treated with PEG-OAC nanozymes or DEF-OAC-PEG nanozymes and an increase in the extracellular acidification rate (ECAR), the rate that protons were released into the media.

The increase in oxygen consumption rate (OCR) suggests an increase in oxidative phosphorylation or some other form of cytosolic oxygen consumption.

We showed in FIG. 2B that the measurement of G2 linked to ATP synthesis increased significantly between the control cells and cells treated with either the PEG-OAC and DEF-OAC-PEG nanozymes, indicating that the nanozyme-treated cells were producing more ATP than when untreated. Another critical endpoint of cellular function is their maximal mitochondrial respiration, a measure of how much oxygen a mitochondrion can consume where we saw a significant increase in OCR (FIG. 2A).

In the context of Friedreich's ataxia, where oxidative phosphorylation is impaired by a lack of iron-sulfur complexes in the mitochondrial complexes, increased maximal respiration is an indicator of increased electron flow from NADH through Complex 1, III, and IV to oxygen which drives the pumping of protons from the mitochondrial matrix to the mitochondrial intermembrane space, consequently increasing ATP production by the return of protons through Complex V In total, an increase in maximal respiration is an indicator of improved mitochondrial respiration.

C. Generation of Cardiomyocytes and Sensory Neurons from FRDA IPSC and Isogenic Controls

FRDA-derived iPSCs were obtained from Dr. Marek Napierala (UT Southwestern) along with isogenic controls in which the GAA frataxin repeats were excised. We successfully differentiated iPSC into cardiomyocytes and FRDA-IPSC sensory neuronal precursors.

### D. Iron Chelation by DEF-OAC-PEG Nanozymes in Cultured Cells

DEF-conjugated PEG-OAC nanozymes are capable of chelating free iron intracellularly as a potential therapeutic tool for resolving elevated iron in the brain following acute neurological insult such as hemorrhagic stroke or traumatic brain injury. In addition, individuals with chronic diseases such as Friedreich's ataxia may also benefit from iron chelation. Without the Fe3+ treatment, the cells are unaffected by the particles suggesting that the particles do not alter iron metabolism in healthy cells.

The iron responsive fluorophore FerroOrange (Dojindo) was used to measure the changes in intracellular iron when bEnd.3 cells treated with 50 mM iron(III) chloride and 4 mg/mL nanozymes, or 100 mM deferoxamine (FIG. 6). Using fluorescence microscopy, we found a significant reduction in fluorescence in cells treated with DEF-OAC-PEG nanozymes or deferoxamine. These findings indicate that DEF-OAC-PEG nanozymes can reduce intracellular iron load.

The concentration of deferoxamine used alone is 100x greater than that of the DEF-OAC-PEG nanozymes. For the reduction in intracellular iron, the DEF-OAC-PEG nanozymes are approximately 63-fold more potent per mole of deferoxamine. This finding suggests that DEF-OAC-PEG nanozymes somehow enhance the chelation ability of deferoxamine.

### II. Mitochondrial Function

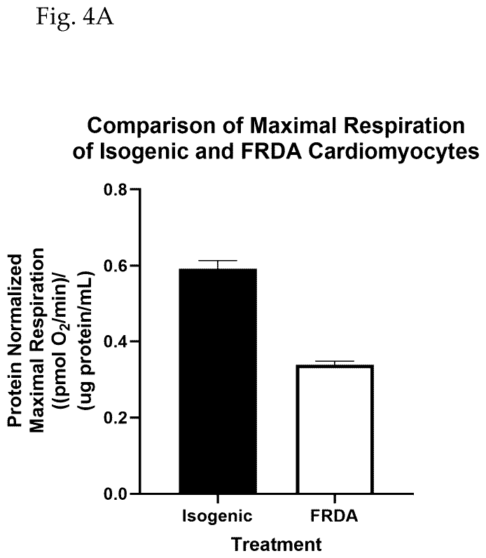

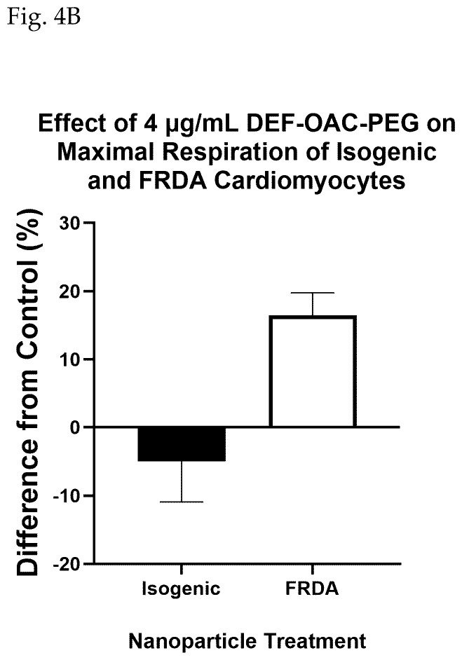

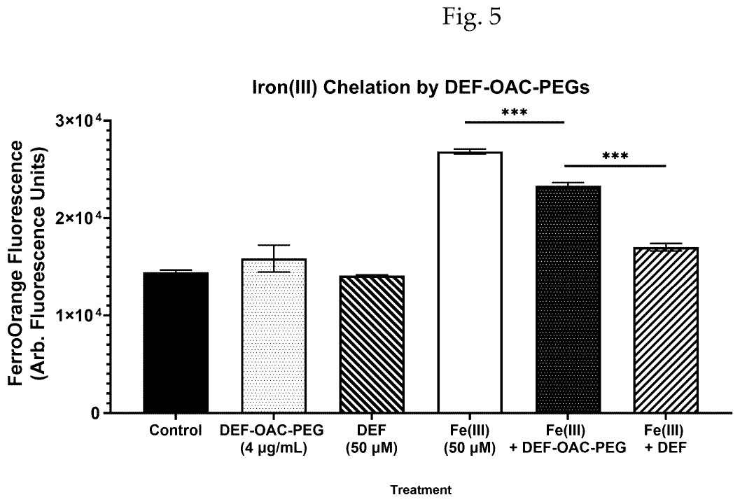

Results utilizing FRDA cardiomyocytes (FIGS. 4A and 4B) demonstrate reduced maximal respiration in FRDA-IPSC-CM compared to isogenic controls (FIG. 4A) and an increase in maximal respiration following a single nanozyme treatment, 4 mg/mL (FIG. 4B) which is not observed in the isogenic line. Quantification of these effects is discussed below, indicating reduced baseline maximal respiration and nanozyme 24 hours exposure with slight reduction in isogenic controls (consistent with our prior data in normal cells).

Comparing the maximal mitochondrial respiration rate of isogenic iPSC-derived cardiomyocytes with human FRDA induced pluripotent stem cell-(iPSC-) cardiomyocytes [FRDA CM] (FIG. 4A) and found that the FRDA maximal rate was 57% of the isogenic rate. Treatment of the FRDA iPSC-cardiomyocytes with the DEF-OAC-PEG nanozymes or 24 hours increased the maximal respiration rate by 15-20% (FIG. 4B). These findings show an increase in mitochondrial oxygen consumption by improving oxidative phosphorylation in the FRDA cardiomyocytes. These results show that mitochondrial respiration is partially restored by treatment with DEF-OAC-PEG nanozymes.

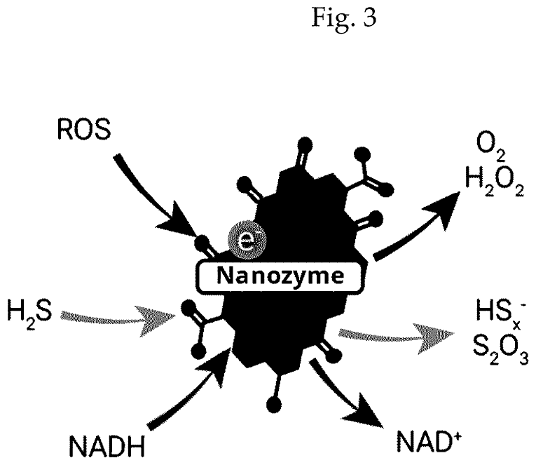

The novel platform technology of PEG-OAC nanozymes with their multifaceted capabilities (FIG. 3) to catalytically dismutate superoxide, enable efficient electron transfer between mitochondrial components, and convert hydrogen sulfide into antioxidant polysulfides is a novel therapeutic approach that holds great promise for mitochondrial diseases and directly relevant to the pathology underlying FRDA.

### III. Reduction in Lipid Peroxidation in FRDA-Derived Fibroblasts

Use of the C11-BODIPY (Dojindo.com) indicates lipid peroxidation (one feature of ferroptosis) by presence of higher green fluorescence relative to red, with the opposite for diminished lipid peroxidation. In FRDA-primary human fibroblast cells, nanozyme treatment induced a predominance of red fluorescence with trends to higher red with the DEF-OAC-PEG nanozymes as would be predicted if iron were involved in lipid peroxidation (FIG. 7).

### IV. PEG-OAC and DEF-OAC-PEG Nanozymes Increase Action Potential in FRDA Cardiomyocytes

Heart failure is a major cause of death in FRDA because of hyperproliferation of faulty mitochondria in cardiomyocytes, enlargement of the heart, and reduction of ejection volume. A key measure of cardiac health is contractility and action potential. Contractility is a measure of how far a cardiomyocyte contracts following an action potential.

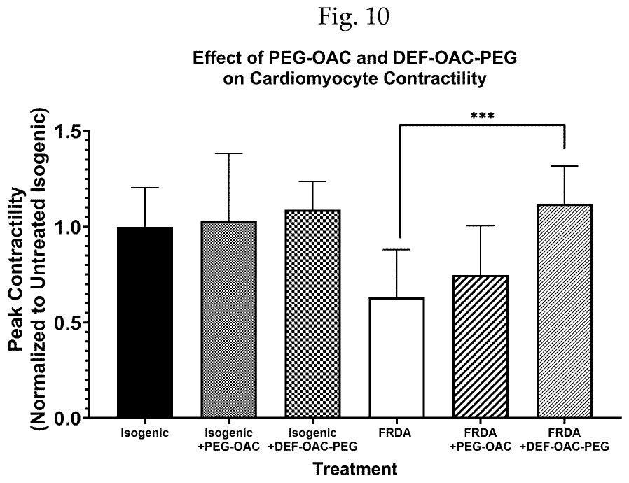

Here we compared isogenic (wild type) inducible-pluripotent stem cell-derived cardiomyocytes with those from an FRDA donor (FIG. 10) and found that the FRDA cells have a peak contractility of 53% of the isogenic cells. A 16% increase in contractility was observed with the PEG-OAC treated cells in the FRDA group and contractility increased to approximately 110% of the isogenic control when treated with DEF-OAC-PEG nanozymes. No significant changes in contractility were observed with the isogenic cardiomyocytes for all treatments. These results are shown in FIG. 10.

The results of this study show that OAC-PEG and DEF-OAC-PEG nanozymes are effective at restoring contractility in FRDA cardiomyocytes. Contractility in myocytes is a direct function of the intracellular availability of ATP which myosin, the molecular motor that facilitates muscle contraction hydrolyzes ATP to change conformations and contract the muscle.

### V. PEG-OAC and DEF-OAC-PEG Nanozymes Reduce Cellular Senescence in FRDA Cells

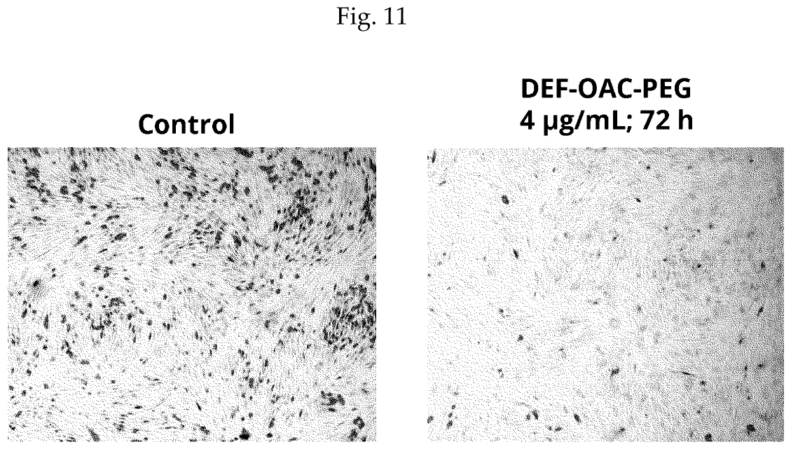

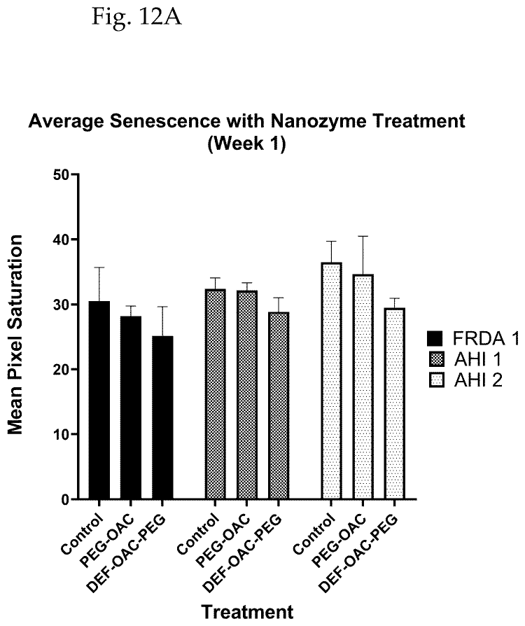

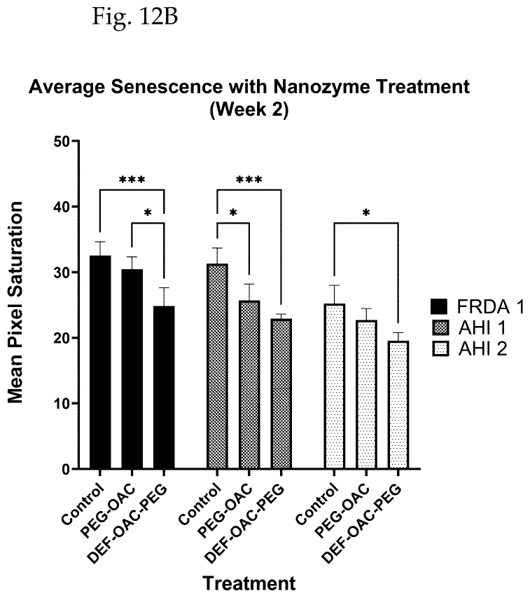

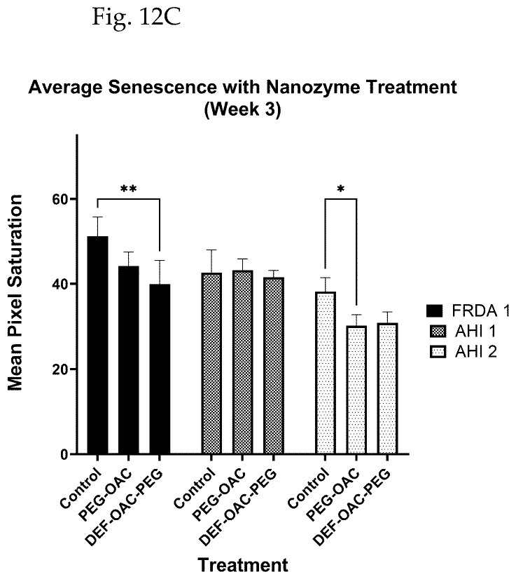

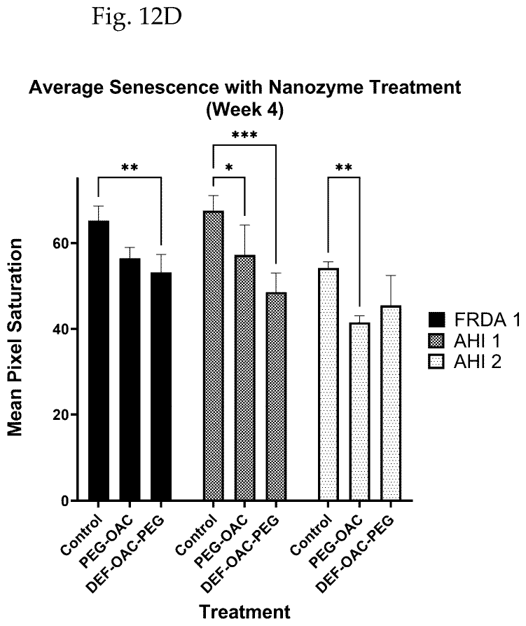

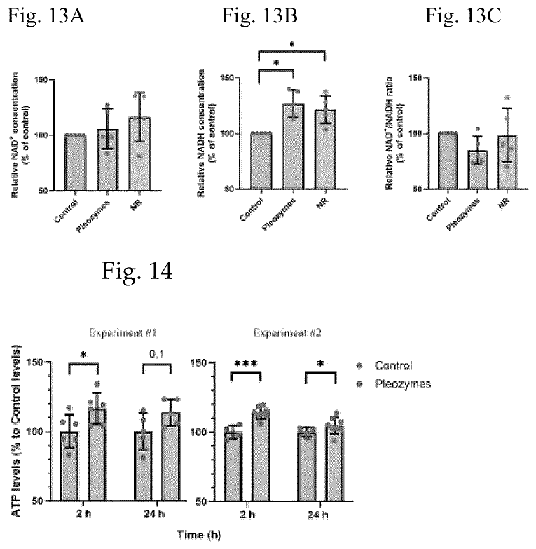

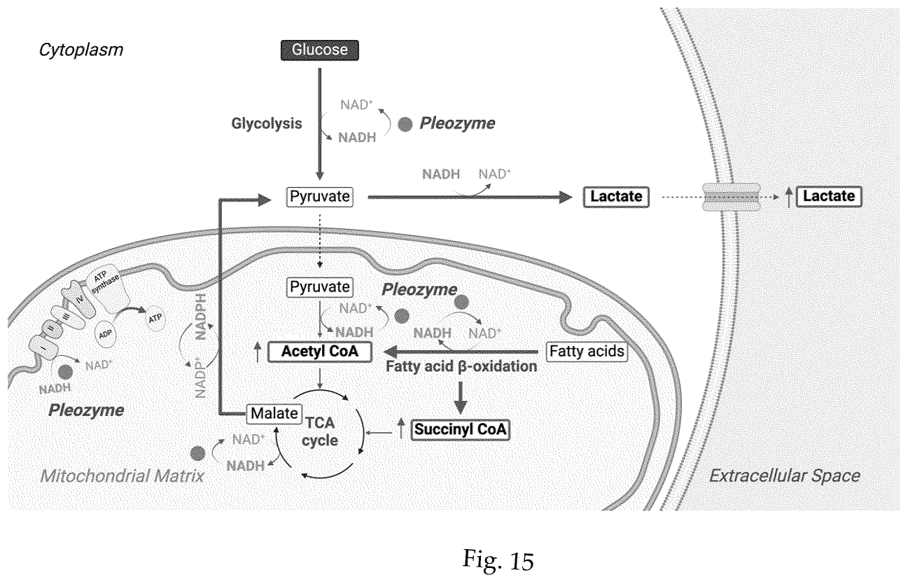

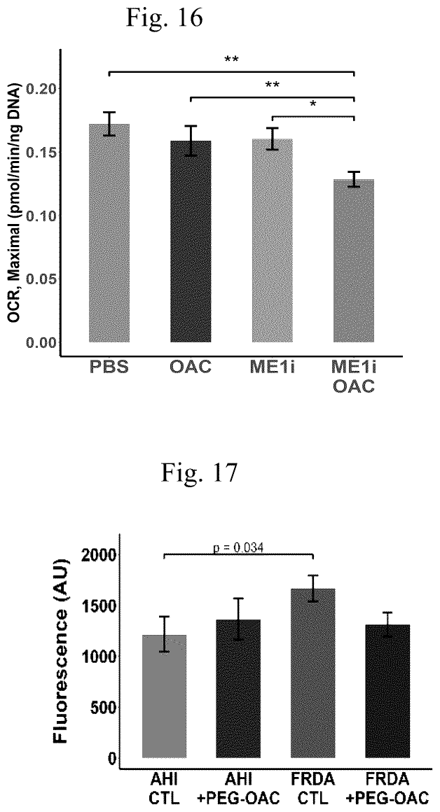

A rapid onset of the senescence phenotype in FRDA skin fibroblasts which was mitigated with a 72-hour exposure to 4 μg/mL DEF-OAC-PEG treatment (FIG. 11) is demonstrated. A longer duration study over the course of four weeks comparing one FRDA cell line to two wild type cell lines (FIGS. 12A, 12B, 12C, and 12D) was performed and a significant reduction in senescence of the FRDA cell line that DEF-OAC-PEG reduced senescence was observed, as measured using X-Gal labeling. In the wild-type cell lines mixed effects were observed, suggesting that the biochemical differences between the two cell types favors the activity of the DEF-OAC-PEG in the FRDA case.

### Pharmaceutical Composition

In one embodiment, the pharmaceutical composition contains a mitochondria-treating effective amount of PEG-OAC, DEF-OAC-PEG or both nanozymes dissolved or dispersed in a physiologically tolerable diluent. In one embodiment, the composition can be a solid, liquid or a gel.

A PEG-OAC nanozyme is typically present as a carboxylic acid and/or carboxylate anion. A cation such as monovalent lithium, sodium, potassium, and ammonium ions, or a divalent ion such as magnesium or calcium can be present to balance the charge of a carboxylate group.

Deferoxamine is a polyamide containing three hydroxamic groups and a primary amine with a molecular weight of 560.6 AMU. As such, after likely bonding its primary amine to carboxyl of a PEG-OAC nanozyme, and the fact that the hydroxamic groups are very weak acids and, that in the absence of metal ions, are completely protonated in acidic or neutral solution, their affinity for the Fe(III) ion is so high that, in an equimolar solution, all of the metal ions are already “seized” at pH 2, a charge-neutralizing companion ion is usually not needed.

In one embodiment, chronic dosing is necessary. The data for the Figs. show that concentrations of about 0.1 to about 2 mg/kg of the recipient, host mammal's weight. In a liquid composition, about 4 mg/mL is about equivalent to the 2 mg/kg dosage. Skilled workers can readily utilize those amounts to find an effective amount for a particular treated subject or cells to be treated.

Administrations are selected from, but not limited to the group consisting of: 1 to about 4 oral doses daily, 1 to about 2 subcutaneous administrations per week, a daily to weekly transdermal application, weekly to biweekly intravenous infusion and combinations therefrom. Stomach acid does not reduce efficacy. Because the contemplated conditions to be treated can be genetic or otherwise require chronic dosing, administrations are likely to continue throughout the mammal's lifetime.

In one embodiment, the pharmaceutical composition is administered orally (perorally) as a liquid or solid formulation. Administration parenterally, is carried out using a liquid pharmaceutical composition. In either situation, a formulation containing conventional nontoxic pharmaceutically acceptable carriers, adjuvants, and vehicles as desired.

The term parenteral as used herein includes subcutaneous injections, intravenous (which is most preferred), intramuscular, intraperitoneal, intrasternal injection, or infusion techniques. Formulation of drugs for oral and parenteral administration is discussed in, standard medicinal chemical texts.

Solid dosage forms for oral administration can include capsules, tablets, pills, powders, and granules. The amount of a contemplated compound in a solid dosage form is an effective amount as discussed previously; and thus provides a concentration to the blood stream of about 0.1 to about 2 mg/kg. A solid dosage form can be and usually is administered a plurality of times during a one-week time period.

In such solid dosage forms, PEG-OAC and/or DEF-OAC-PEG nanozymes are ordinarily combined with one or more diluents appropriate to the indicated route of administration. If administered per os, the compounds can be admixed with lactose, sucrose, starch powder, cellulose esters of alkanoic acids, carboxycellulose, carboxycellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration.

Solid dosage forms such as capsules or tablets can contain a controlled-release formulation as can be provided in a dispersion of contemplated oxidized carbon nanoparticulate material active agent in hydroxypropylmethyl cellulose. In the case of capsules, tablets, and pills, the dosage forms can also include buffering agents such as sodium citrate, magnesium or calcium carbonate or bicarbonate. Tablets and pills can additionally be prepared with enteric coatings for passage through the stomach without release of the contemplated oxidized carbon nanoparticulate material.

In one embodiment, the pharmaceutical composition can also be adapted for parenteral administration. Thus, a pharmaceutical composition is preferably in liquid form when administered, and most preferably, the liquid is an aqueous liquid, although other liquids are contemplated as discussed below, and a presently most preferred composition is an injectable preparation.

Thus, injectable preparations, for example, sterile injectable aqueous or oleaginous solutions or suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol.

Among the acceptable vehicles and solvents that can be employed are water, Ringer's solution, and isotonic sodium chloride solution, phosphate-buffered saline. Sterile solutions can be prepared by dissolving the active component in the desired solvent system, and then passing the resulting solution through a membrane filter to sterilize it or, alternatively, by dissolving the sterile compound in a previously sterilized solvent under sterile conditions.

Other liquid pharmaceutical compositions include, for example, solutions suitable for parenteral administration. Sterile water solutions containing a nanozyme active component as discussed herein or sterile solution of the active component nanozyme in solvents comprising water, ethanol, or propylene glycol are examples of liquid compositions suitable for parenteral administration. In one embodiment, a contemplated nanozyme is provided as a dry powder that is to be dissolved (dispersed) in an appropriate liquid medium such as sodium chloride for injection prior to use.

It is also to be understood that in therapeutic formulations the active oxidized carbon nanoparticulate materials can be dispersed, dissolved, emulsified, suspended, lyophilized, encapsulated, micronized, nanoparticulated and the like. In addition, the formulations can contain solvents such as alcohols, polyols, esters, water; thickening and consistency modulating agents such as galactomannan gums, carbohydrate polymers such as starch, cellulose derivatives, alginic acid and derivatives, alkyl acrylate polymers and copolymers, polyvinyl alcohol and derivatives; film forming agents such as cellulose ethers, carbomers; chelating agents, emulsifying agents such as long chain fatty acids, regular or fatty alcohols, and or esters; amino acids, and buffering agents.

A stable lyophilized pharmaceutical composition as contemplated herein can be made by lyophilizing a solution comprising nanozyme material alone or admixed with a bulking agent, e.g., mannitol, trehalose, raffinose, and sucrose or mixtures thereof. There are many other conventional lyophilizing agents. Among the sugars, lactose is the most common. Also used are citric acid, sodium carbonate, EDTA, benzyl alcohol, glycine, sodium chloride, etc.

In addition, sterile, fixed oils are conventionally employed as a solvent or suspending medium. For this purpose, any bland, sterile fixed oil can be employed including synthetic mono- or diglycerides. In addition, fatty acids such as oleic acid find use in the preparation of an injectable composition. Dimethyl acetamide, surfactants including ionic and non-ionic detergents, polyethylene glycols can be used. Mixtures of solvents and wetting agents such as those discussed above are also useful.

A host mammal in need of treatment (a subject) and to which a pharmaceutical composition containing a contemplated nanozyme is administered is typically a human. Other, non-human primates such as an ape such as a chimpanzee or gorilla, a monkey such as a cynomolgus monkey or a macaque, a laboratory animal such as a rat, mouse or rabbit, a companion animal such as a dog, cat, horse, or a food animal such as a cow or steer, sheep, lamb, pig, goat, llama or the like can also be used as test subjects in toxicity assays and the like.

Preferably, a pharmaceutical composition is in unit dosage form. In such form, the composition is divided into unit doses containing appropriate quantities of the oxidized carbon nanoparticulate material active compound. The unit dosage form can be a packaged preparation, the package containing discrete quantities of the preparation, for example, in vials or ampules.

### Nanozyme Preparation

The contemplated nanozymes are prepared as described in FIG. 1. In brief, the nanozymes described herein are composed of an oxidized activated charcoal core made from good manufacturing practice coconut husks (OAC) and functionalized with poly(ethylene glycol)-NH₂ (PEG-NH₂) and the iron chelator deferoxamine (DEF), to the OAC core particle PEG-NH₂ and DEF are conjugated to the carboxyl groups (C—OOH) via amide coupling. (FIG. 1A). The core particles are planar and are approximately 3 nm in diameter (FIGS. 1B, 1C). Thermogravimetric analysis showed a reduction in mass loss DEF-OAC-PEG compared to PEG-OAC indicating that they carried less PEG-NH₂ (FIG. 1D).

The hydrodynamic radii of the DEF-OAC-PEG was larger than PEG-OAC (94 vs 89 nm) (FIG. 1E), suggesting that the particles interacted more strongly, likely due to a reduction in PEG. X-ray photoelectron spectroscopy of the core, PEG-cOACs, and DEF-OAC-PEG (FIG. 1F-1H; Table 1) have distinct features but suggest that the particles are decorated with C—OH, C═O, C—OOH, and C—O—C functional groups with a largely sp2-conjugated carbon core.

### XPS Analysis

Elemental composition and oxygen-functionality distribution of OACs, PEG-OACs and DEF-OAC-PEGs were calculated from XPS survey and high-resolution C 1s peak measurements. Those results are shown below in Table 1, in which each value is reproducible within ±5%.

TABLE 1

Atom percentage (at %)

| Functional | OAC | PEG-OACs | DEF-OAC- |
|---|---|---|---|
| Group | (at %) | (at %) | PEGs (at %) |
| C═C/C—C | 66.2 | 7.9 | 16.3 |
| C—O—C/C—O | 12.1 | 79.9 | 70.0 |
| C═O | 6.6 | 6.5 | 9.4 |
| O—C═O | 15.2 | 5.8 | 4.2 |
| N | 0 | 0.6 | 1.7 |
| C:O Ratio | 2.6 | 1.9 | 2.1 |

X-Ray photoelectron spectroscopy (Table 1) showed that DEF-OAC-PEGs had less C—O content (70.0 vs 79.9%) indicative of DEF covalently bound to the nanoparticle indicative of less PEG. The C—OOH content was lower in the DEF-OAC-PEGs, which suggested a higher degree of C—OOH functionalization. This follows because PEG is a bulky molecule whereas DEF is small and is not as susceptible to steric hindrance during functionalization. The content of nitrogen in the functionalized samples was different as well and suggests that the DEF-OAC-PEGs are more completely functionalized than the PEG-OACs. This is bolstered in part by the 6 N atoms per molecule of DEF rather than the single N atom in PEG-NH₂.

Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity, such as sodium chloride or dextrose. The pH can be adjusted with acids or bases, such as hydrochloric acid or sodium hydroxide. The preparation can be enclosed in ampoules, disposable syringes or multiple-dose vials made of glass or plastic. For convenience of the patient or treating physician, the dosing formulation can be provided in a kit containing all necessary equipment (e.g., vials of drug, vials of diluent, syringes and needles) for a course of treatment (e.g., 7 days of treatment).

Sterile injectable solutions can be prepared by incorporating the active chemical compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

The preferred route may vary with, for example, the subject's pathological condition or age or the subject's response to therapy or that is appropriate to the circumstances. The formulations can also be administered by two or more routes, where the delivery methods are essentially simultaneous, or they may be essentially sequential with little or no temporal overlap in the times at which the composition is administered to the subject.

Suitable regimes for initial administration and further doses or for sequential administrations also are variable, may include an initial administration followed by subsequent administrations, but nonetheless, may be ascertained by the skilled artisan from this disclosure, the documents cited herein, and the knowledge in the art.

### Miscellaneous

As used herein, the phrase “effective amount” shall mean that dosage that provides the specific immunological response for which a composition comprising that effective amount is administered in a significant number of subjects. For example, an effective amount of an antigen may include that amount which when administered to a vaccinee induces an immune response in the vaccinee, preferably a protective immune response against the pathogen from which the antigen is derived. An effective amount of an antigen that is administered to a particular patient in a particular instance will not always be effective in treating the conditions/diseases described herein, even though such dosage is deemed to be a therapeutically effective amount by those of skill in the art.

The compositions disclosed herein optionally include an adjuvant. The term “adjuvant” refers to a compound or mixture that enhances an immune response.

As used herein, the term “administering”, refers to dispensing, delivering, or applying the therapeutic agent, to a subject by any suitable route for delivery of the substance to the desired location in the subject, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

The terms “treating” and “to treat” includes the reducing, repressing, delaying or preventing inherited mitochondrial disease.

As used herein, the term “subject” may be used interchangeably with the term “patient” or “individual” and may include an “animal” and in particular a “mammal.” Mammalian subjects may include humans and non-human animals, such as other primates, domestic animals, farm animals, and companion animals such as dogs, cats, guinea pigs, rabbits, rats, mice, horses, cattle, cows, etc.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of” those certain elements.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The foregoing description and the examples are intended as illustrative and are not to be taken as limiting. Still other variations within the spirit and scope of this invention are possible and will readily present themselves to those skilled in the art.

1. A method for treating a mammal exhibiting an inherited mitochondrial disease comprising administering a pharmaceutical composition containing a mitochondria-treating effective amount of PEG-OAC nanozymes, DEF-OAC-PEG nanozymes or both nanozymes dissolved or dispersed in a physiologically tolerable diluent, wherein PEG is an acronym for a reacted alpha-amino-omega-methoxy-poly(ethylene glycol) substituent, OAC is an acronym for oxidized activated charcoal particle, and DEF is an acronym for a reacted deferoxamine substituent, and wherein said PEG and said DEF substituents are each covalently bonded to said OAC by the primary amino group on each.

2. The method according to claim 1, wherein said PEG has an average molecular weight of about 2000 to about 10,000 Da.

3. The method according to claim 1, wherein said PEG has an average molecular weight of about 5000 Da.

4. The method according to claim 1, wherein said PEG-OAC nanozymes contain an average of about 2 to about 5 of said reacted PEG substituents per particle, an average of 2 to about 5 PEG groups chemically bonded to the particle.

5. The method according to claim 1, wherein said PEG-OAC-DEF nanozymes contain an average of about 2 to about 5 PEG substituents per particle.

6. The method according to claim 1, wherein said PEG-OAC-DEF nanozymes contain an average of about 2 to about 5 PEG substituents per and about 10 to about 20 DEF substituents per particle.

7. The method according to claim 1, wherein said PEG-OAC-DEF nanozymes contain an average of about the same number of PEG substituents and DEF substituents per particle.

8. The method according to claim 1, wherein physiologically tolerable diluent is an aqueous liquid adapted for parenteral administration.

9. The method according to claim 8, wherein said aqueous liquid is Ringer's solution, isotonic sodium chloride solution or phosphate-buffered saline.

10. The method according to claim 1, wherein said nanozymes are present in an amount of about 0.1 to about 2 mg/kg.

11. The method according to claim 1, wherein only one of said nanozymes is present.

12. The method according to claim 1, wherein said physiologically acceptable diluent is adapted for oral administration.

13. The method according to claim 12, wherein said physiologically acceptable diluent is an aqueous liquid.

14. The method according to claim 11, wherein said physiologically acceptable diluent is a solid.

15. The method according to claim 1, wherein said administration is repeated.

16. The method according to claim 1, wherein said mammal is a human.

17. The method according to claim 1, wherein said mammal is a non-human primate, a laboratory animal, a companion animal, or a food animal.

18. The method according to claim 1, wherein said inherited mitochondrial disease is Friedreich's ataxia.

19. A pharmaceutical composition containing a mitochondria-treating effective amount of PEG-OAC nanozymes, DEF-OAC-PEG nanozymes or both nanozymes dissolved or dispersed in a physiologically tolerable diluent, wherein PEG is an acronym for a reacted alpha-amino-omega-methoxy-poly(ethylene glycol) substituent, OAC is an acronym for oxidized activated charcoal particle, and DEF is an acronym for a reacted deferoxamine substituent, and wherein said PEG and said DEF substituents are each covalently bonded to said OAC by the primary amino group on each.

20. A method for treating a mitochondrial disease or disorder comprising administering to a patient in need a pharmaceutical composition comprising a compound selected from the group consisting of: a mitochondria-treating effective amount of PEG-OAC nanozymes, DEF-OAC-PEG nanozymes and combinations thereof, wherein said nanozymes are dissolved or dispersed in a physiologically tolerable diluent.
